(19) United States
(12) Patent Application Publication
(10) Pub. No.: US 2019/0321574 A1
(43) Pub. Date: Oct. 24, 2019

(54) AUTOMATIC SUPPLEMENTAL OXYGEN CONTROL SYSTEM WITH WEANING CAPABILITIES

(71) Applicant: AutO2, LLC, Glendale, MO (US)

(72) Inventors: Christopher Sallee, St. Louis, MO (US); Alex Wisbeck, St. Louis, MO (US); Brian Albers, St. Louis, MO (US); Chad Chapnick, St. Louis, MO (US); Peter Iliya, St. Louis, MO (US)

(21) Appl. No.: 16/392,272

(22) Filed: Apr. 23, 2019

Related U.S. Application Data
(60) Provisional application No. 62/661,453, filed on Apr. 23, 2018.

(51) Int. Cl. A61M 16/00; A61B 5/087; A61B 5/00

(52) U.S. Cl. CPC A61M 16/024; G16H 40/67; A61B 5/743; A61B 5/087

(57) ABSTRACT

Disclosed herein is a device and method for delivery of supplemental oxygen in a healthcare environment such that flow rate of delivered oxygen is dependent on input data of patient oxygen saturation values and other physiologic variables. The output of oxygen is modulated by an algorithm which may be pre-set or altered by the user in which variables such as desired range of oxygen saturation, oxygen flow rate of change, time response and alarm settings may be altered. User interaction may be accomplished via interface on the device itself comprising of a touchscreen, buttons or dial system or via interaction with a second device such as a patient vitals monitor or computer, which may interact with the device by direct connection or network connection. The device may be used to maintain a state of adequate oxygenation in the patient by increasing or decreasing oxygen flow, or to facilitate weaning and cessation of oxygen use.

### CROSS REFERENCE TO RELATED APPLICATIONS

This application claims priority to U.S. Provisional Application No. 62/661,398, filed Apr. 23, 2018, the contents of which are entirely incorporated by reference herein.

### FIELD

The present disclosure relates to an automatic supplemental oxygen control system with weaning capabilities.

### BACKGROUND

In a healthcare setting, supplemental oxygen is administered to patients, particularly to neonates or patients with respiratory complaints. The level of oxygen in blood is measured using a pulse oximeter, which clips onto the patient's finger and uses a photodiode to measure the amount of hemoglobin bound to oxygen in arterial blood, denoted as SpO₂. Unless saturation levels fall below a threshold and sounds an alarm to alert healthcare staff, adjustment of patient oxygen occurs primarily during scheduled visits by nurses or other healthcare staff to adjust other aspects of the patient's healthcare regimen. The current method for oxygen delivery can be significantly improved by implementing a closed-loop system which adjusts oxygen output dependent upon a patient's SpO₂ and is capable of incrementally decreasing oxygen administered to the patient in order to facilitate more efficient weaning off of supplemental oxygen. Existing closed-loop systems fail to combine features related to (1) automated control of oxygen flow or FiO₂ output in regards to a measure of patient oxygenation, most commonly SpO₂, (2) network connectivity such that settings of the device may be altered by an authorized user by a secondary system such as a computer or application on the network, (3) settings designed to facilitate efficient weaning off of supplemental oxygen and (4) a method of airflow regulation contained within the system.

Therefore, there is a need for a device and method for automatically controlling oxygen flow, having network connectivity, facilitate efficient weaning of supplemental oxygen, and regulating airflow within the system.

### SUMMARY

The disclosure provides a device for automatically adjusting the flow of oxygen delivered to a patient setting may include a microcontroller and a delivery system for automatically incrementally increasing or decreasing the amount of oxygen delivered to the patient by modifying the flow of oxygen to the patient and subsequently inspired fraction of oxygen. The device may further include a user interface, a secondary device connector, and a system of alarms.

The microcontroller may include a processing module for processing patient input data comprising oxygen saturation data, and an adjustment module for calculating the adjustment of oxygen output to the patient. The adjustment of oxygen may be calculated using the patient input data of the processing module. The amount of oxygen delivered may be based on the adjustment of oxygen calculated in the adjustment module.

Further provided herein is a method for automatically adjusting the flow of oxygen delivered to a patient. The method may include processing patient input data comprising oxygen saturation data, calculating the adjustment of oxygen output to the patient, and automatically incrementally increasing or decreasing the amount of oxygen delivered to the patient by modifying the flow of oxygen to the patient and subsequently inspired fraction of oxygen. The adjustment of oxygen may be calculated using the patient input data. The amount of oxygen delivered may be based on the calculated adjustment of oxygen.

Additional embodiments and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

### BRIEF DESCRIPTION OF DRAWINGS

The description will be more fully understood with reference to the following figures and data graphs, which are presented as various embodiments of the disclosure and should not be construed as a complete recitation of the scope of the disclosure, wherein:

FIG. 1 is an overall depiction of the device framework and integration with other components such as the oxygen supply and LAN, which could possibly comprise patient monitors and EMR servers.

FIG. 2 highlights the device's Feedback Controller responsible for automation concerning patient oxygen stabilization and/or patient oxygen cessation.

FIG. 3 details how inputs of Feedback Controller are processed internally to decide the oxygen flow to be administered to the patient, whether patient oxygen stabilization and/or patient oxygen cessation.

FIG. 4 isolates and describes the parameters and their associated values of PID controller system H1(t).

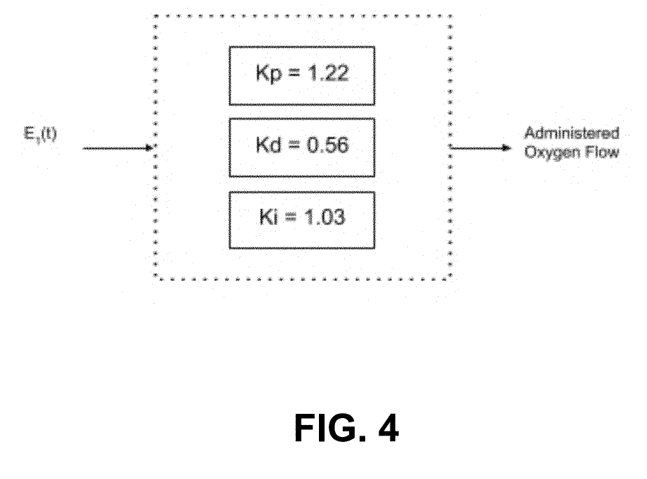

FIG. 5 highlights the device's Oxygen Flow Controller system and inputs responsible for ensuring accurate administered oxygen flow.

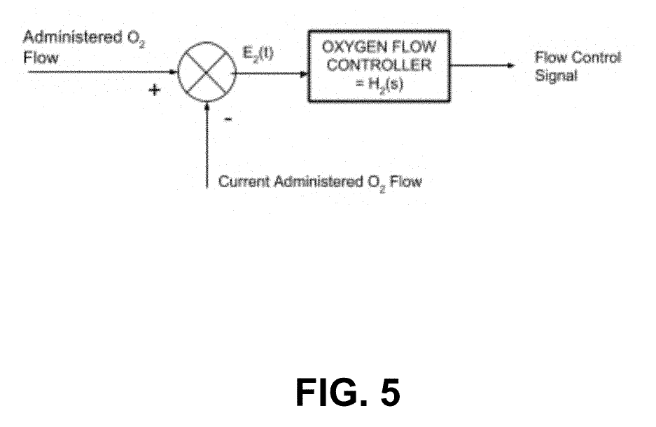

FIG. 6 isolates and describes the parameters and their associated values of PID controller system H2(t).

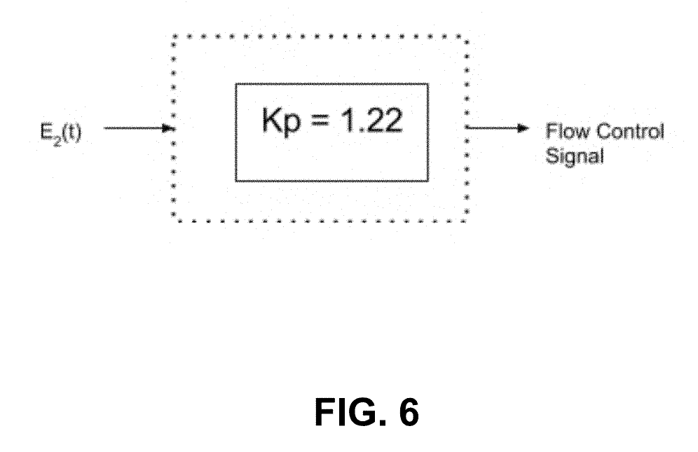

### DETAILED DESCRIPTION

The disclosure may be understood by reference to the following detailed description, taken in conjunction with the drawings as described below. It is noted that, for purposes of illustrative clarity, certain elements in various drawings may not be drawn to scale.

Provided herein is a device and method for delivery of supplemental oxygen in a healthcare environment such that flow rate of delivered oxygen is dependent on input data of patient oxygen saturation values and other physiologic variables. The output of oxygen is modulated by an algorithm which may be pre-set or altered by the user in which variables such as desired range of oxygen saturation, oxygen flow rate of change, time response and alarm settings may be altered. User interaction may be accomplished via interface on the device itself including a touchscreen, buttons or dial system or via interaction with a second device such as a patient vitals monitor or computer, which may interact with the device by direct connection or network connection. The device may be used to maintain a state of adequate oxygenation in the patient by increasing or decreasing oxygen flow, or to facilitate weaning and cessation of oxygen use.

### Automatic Adjustment Device

The device for automatically adjusting the flow of oxygen delivered to a patient setting may include a microcontroller and a delivery system operable for automatically incrementally increasing or decreasing the amount of oxygen delivered to the patient by modifying the flow of oxygen to the patient and subsequently inspired fraction of oxygen. The device may further include a user interface, a secondary device connector, and/or a system of alarms.

The microcontroller may include a processing module for processing patient input data comprising oxygen saturation data, an adjustment module for calculating the adjustment of oxygen output to the patient, and/or an alarm module for sounding an alarm if the oxygen saturation data fall below a threshold. The adjustment of oxygen may be calculated using the patient input data of the processing module. The amount of oxygen delivered may be based on the adjustment of oxygen calculated in the adjustment module.

The secondary device connector may include, but is not limited to, a physical connector and a wireless network connector. Non-limiting examples of the secondary device include a monitoring system, a computer, a tablet, or a mobile device. The microcontroller may further include a correction module for the identification and correction of artifacts in the patient input data. The oxygen saturation data may be obtained from a pulse oximeter or transcutaneous membrane, and the patient's heart rate may be obtained from the pulse oximeter. The patient input data may further include, but is not limited to, heart rate, respiratory rate, expiratory CO₂ levels, blood pressure and/or temperature. The patient input data may be obtained directly from the measuring device, from a monitoring system, or over a LAN network.

In an aspect, the delivery system may provide automatic oxygen weaning and facilitate eventual cessation of oxygen delivery to the patient. Adequate oxygen saturation of the patient may be maintained by automatic adjustments in oxygen delivery. The delivery system may include, but is not limited to, at least one valve, at least one motor, a solenoid system, and combinations thereof. In an aspect, the delivery system may be modulated internally or by manual adjustment, and the delivery system may be manually overridden. The device may receive oxygen from a system such as, but not limited to, a wall output, oxygen cylinder compressor and a reservoir. Oxygen may be delivered to the patient by a nasal cannula or a facemask.

The user interface of the device may include a user input mechanism operably connected to the microcontroller for altering at least one parameter of the device. For example, the user input mechanism may be configured for entering or altering the patient input data, the adjustment of oxygen, the threshold, and/or the amount of oxygen delivered to the patient. In some aspects, the user interface further includes a graphical representation of the patient's oxygen saturation and device oxygen output over time, a graphical representation of patient physiologic data, a graphical representation of data in real time, and a graphical representation of historical data in order to monitor the course of a patient's care or the device's performance over time. In some examples, the user interface includes a representation or input for modifying the parameters of the processing module or adjustment module.

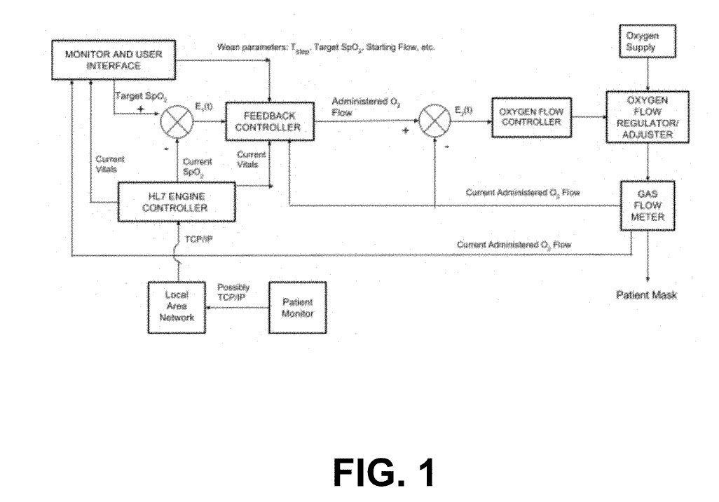

In various aspects, the device may interface with medical oxygen supplies, HL7 data over a local area network, servers over a network, SpO₂ sensors, or supplemental oxygen applicators and equipment. In an aspect, it may be located within patient rooms, upright at eye-level, and easily mobile. In other aspects, the device may be located at any location within the patient's room. In further aspects, the device may not be located in the patient's room and remotely connect to the secondary devices. In order to process HL7 data the device may include an HL7 engine or library seen in FIG. 1, which may also convert any outgoing patient data into HL7 before being sent out to the electronic medical record (EMR).

Additionally, the device may include house ports for connecting oxygen air supplies at about 50 psi to about 55 psi. As seen in FIG. 1 this may require an initial regulator similar to the flow regulators connected to wall air supplies in hospitals, but does not have to be limited this one purpose.

The device may be used to monitor, report to an EMR, and autonomously regulate a patient's administered flow of supplemental oxygen based off of SpO₂ readings. Users of the device may specify how the device performs the autonomous regulation by inputting relevant parameters' values into the device using device graphic user interface (GUI) at any point in time. This GUI may also in real-time display the current progress and/or history of this regulation in terms of SpO₂ and oxygen flow over time.

### Automatic O₂ Adjustment Method

Further provided herein is a method for automatically adjusting the flow of oxygen delivered to a patient. The method may include processing patient input data comprising oxygen saturation data, calculating the adjustment of oxygen output to the patient, and automatically incrementally increasing or decreasing the amount of oxygen delivered to the patient by modifying the flow of oxygen to the patient and subsequently inspired fraction of oxygen. The adjustment of oxygen may be calculated using the patient input data. The amount of oxygen delivered may be based on the calculated adjustment of oxygen.

The method may further include connecting with a secondary device. The secondary device may be connected by a physical connection or a wireless network. The secondary device may include, but is not limited to, a monitoring system, a computer, a tablet, or a mobile device. In an aspect, this may further include identifying and correcting artifacts in the patient input data. The oxygen saturation data may obtained from a pulse oximeter or transcutaneous membrane, and the patient's heart rate may be obtained from the pulse oximeter. Non-limiting examples of the patient input data include heart rate, respiratory rate, expiratory CO₂ levels, blood pressure and temperature. The patient input data may be obtained directly from the measuring device, from a monitoring system or over a LAN network.

In an aspect, the method may further include automatically weaning oxygen and facilitating eventual cessation of oxygen delivery. Adequate oxygen saturation of the patient may be maintained by automatic adjustments in oxygen delivery. The amount of oxygen delivered to the patient may be increased or decreased by a delivery system including, but not limited to, at least one valve, at least one motor, a solenoid system, and combinations thereof. In various aspects, the delivery system may be modulated internally or by manual adjustment, and the delivery system may be manually overridden. The method may further include receiving oxygen from a system including, but not limited to, a wall output, oxygen cylinder compressor and a reservoir. In an aspect, oxygen may be delivered to the patient by a nasal cannula or a facemask.

The method may further include identifying and correcting input data artifacts. In an aspect, the signal of heart rate from the pulse oximeter may be used to assess the validity of the oxygen saturation data. The method may further include alerting the user of adverse events such as prolonged oxygen saturation in the hypoxic range or hyperoxic range, expiratory CO₂ levels outside the target range, loss of data signal, presence of data artifacts, loss or changes to network connection, mechanical dysfunction of the device, or other adverse events. The loss of data signal may correspond to the pulse oximeter or transcutaneous membrane losing contact with the patient.

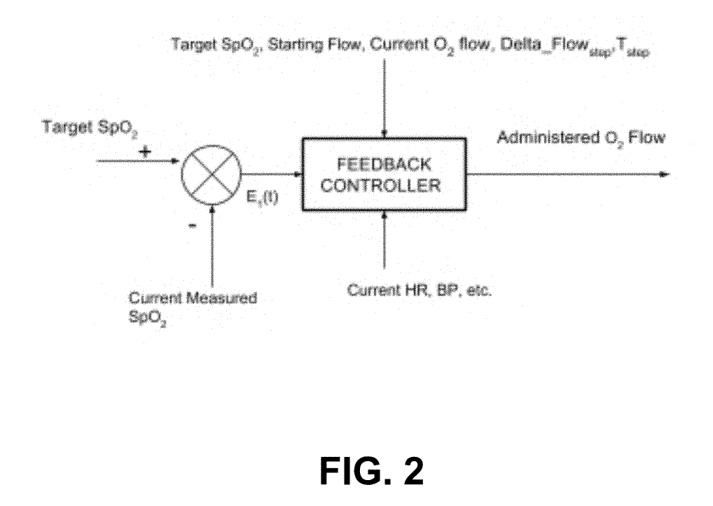

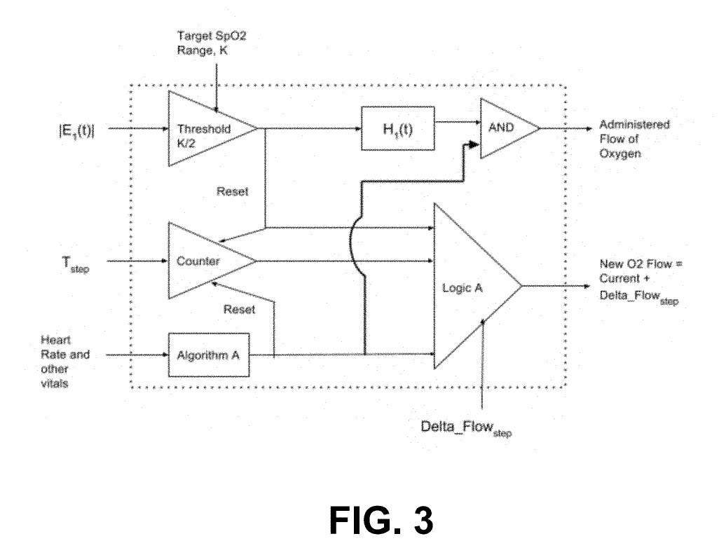

Autonomous regulation of a patient's administered supplemental oxygen flow is illustrated by FIG. 2 and may occur in two independent, but not exclusive modes: stabilization and cessation. Stabilization is when the device autonomously keeps a patient within a SpO₂ range indefinitely by changing the oxygen flow according to control system H₁(s) seen in FIG. 3. Cessation or weaning is when the patient is weaned off of oxygen at user specified time increments (Tₛₜₑₚ) by drops in oxygen flow specified by Delta_Flowₛₜₑₚ.

These independent modes may be controlled by the device, for example control system H₁(s) and the rules of Logic A as seen in FIG. 3. Only when the patient's SpO₂ has gone out of the specified Target SpO₂ Range (K) will oxygen flow be autonomously adjusted according to control function H₁(s). Thus the purpose of H₁(s) is to control oxygen flow in order to stabilize or re-stabilize a patient to the user-set range of SpO₂. This feature not only presents a fail-safe in the event of unforeseen dramatic changes in patient SpO₂, but ensures precise and optimized weaning protocols.

When the patient is within the target SpO₂ range, the control system H₁(s) is not enabled and the counter is enabled. The counter is responsible for keeping track of each weaning step in a weaning/cessation protocol. If the counter reaches Tstep and the data during this last Tstep amount of time was valid by Algorithm A and did not trigger the threshold K, then by Logic A the oxygen flow is decremented by delta_Flow. The truth table for all possible states seen by Logic A is shown in Table 1.

| | | Other Physiological | |
|---|---|---|---|
| E(t) | Tstep | Vitals | Result of Logic A |
| False | False | False | No change. (Counter is reset) |
| False | False | True | No change. |
| False | True | False | No change. (Counter is reset) |
| False | True | True | Adjust flow of oxygen by |
| | | | Delta_Flowₛₜₑₚ |
| True | False | False | No change. (Counter is reset) |
| True | False | True | No change. (Counter is reset) |
| True | True | False | No change. (Counter is reset) |
| True | True | True | No change. (Counter is reset) |

Note that for any oxygen flow change to occur autonomously the incoming data must be proved valid by Algorithm A, which uses all patient vitals to ensure data reliability and robustness. Whenever data is invalid (for a significant amount time dictated by Algorithm A) or whenever SpO₂ goes out of range, the counter in FIG. 3 is reset to ensure that patient has fully stabilized to a stable FiO₂ for Tstep amount of time before another decrement in the oxygen flow is made.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

Numerous examples are provided herein to enhance the understanding of the present disclosure. A specific set of statements are provided as follows.

Statement 1: A device for automatically adjusting the flow of oxygen delivered to a patient setting comprising a microcontroller comprising a processing module for processing patient input data comprising oxygen saturation data; an adjustment module for calculating the adjustment of oxygen output to the patient, wherein the adjustment of oxygen is calculated using the patient input data of the processing module; and an alarm module; a delivery system for automatically incrementally increasing or decreasing the amount of oxygen delivered to the patient by modifying the flow of oxygen to the patient and subsequently inspired fraction of oxygen, wherein the amount of oxygen delivered is based on the adjustment of oxygen calculated in the adjustment module; a user interface; and a secondary device connector.

Statement 2: The device of Statement 1, wherein the secondary device connector is selected from a physical connection and a wireless network.

Statement 3: The device of Statement 1, wherein the secondary device is a monitoring system, a computer, a tablet, or a mobile device.

Statement 4: The device of Statement 1, wherein the microcontroller further comprises a correction module for the identification and correction of artifacts in the patient input data.

Statement 5: The device of Statement 1, wherein the oxygen saturation data is obtained from a pulse oximeter or transcutaneous membrane.

Statement 6: The device of Statement 5, wherein the patient's heart rate is obtained from the pulse oximeter.

Statement 7: The device of Statement 1, wherein the patient input data further comprises the patient's data selected from heart rate, respiratory rate, expiratory CO2 levels, blood pressure and temperature.

Statement 8: The device of Statement 1, wherein the patient input data is obtained directly from the measuring device, from a monitoring system or over a LAN network.

Statement 9: The device of Statement 1, wherein the delivery system provides automatic oxygen weaning and facilitates eventual cessation of oxygen delivery.

Statement 10: The device of Statement 9, wherein adequate oxygen saturation of the patient is maintained by automatic adjustments in oxygen delivery.

Statement 11: The device of Statement 1, wherein the delivery system is selected from at least one valve, at least one motor, a solenoid system, and combinations thereof.

Statement 12: The device of Statement 1, wherein the delivery system may be modulated internally or by manual adjustment, and wherein the delivery system may be manually overridden.

Statement 13: The device of Statement 1, wherein the device receives oxygen from a system selected from a wall output, oxygen cylinder compressor and a reservoir.

Statement 14: The device of Statement 1, wherein oxygen is delivered to the patient by a nasal cannula or a facemask.

Statement 15: The device of Statement 1, wherein the user interface comprises a user input mechanism operably connected to the microcontroller; a graphical representation of the patient's oxygen saturation and device oxygen output over time; a graphical representation of patient physiologic data; a graphical representation of data in real time; and a graphical representation of historical data in order to monitor the course of a patient's care or the device's performance over time.

Statement 16: The device of Statement 15, wherein the user input mechanism is configured for entering or altering the patient input data, the adjustment of oxygen, the threshold, and/or the amount of oxygen delivered to the patient.

Statement 17: A method for automatically adjusting the flow of oxygen delivered to a patient comprising processing patient input data comprising oxygen saturation data; calculating the adjustment of oxygen output to the patient, wherein the adjustment of oxygen is calculated using the patient input data; and automatically incrementally increasing or decreasing the amount of oxygen delivered to the patient by modifying the flow of oxygen to the patient and subsequently inspired fraction of oxygen, wherein the amount of oxygen delivered is based on the calculated adjustment of oxygen.

Statement 18: The method of Statement 17 further comprising connecting with a secondary device.

Statement 19: The method of claim 18, wherein the secondary device is connected by a physical connection or a wireless network.

Statement 20: The method of Statement 19, wherein the secondary device is a monitoring system, a computer, a tablet, or a mobile device.

Statement 21: The method of Statement 17 further comprises identifying and correcting artifacts in the patient input data.

Statement 22: The method of Statement 17, wherein the oxygen saturation data is obtained from a pulse oximeter or transcutaneous membrane.

Statement 23: The method of Statement 22, wherein the patient's heart rate is obtained from the pulse oximeter.

Statement 24: The method of Statement 17, wherein the patient input data further comprises the patient's data selected from heart rate, respiratory rate, expiratory CO2 levels, blood pressure and temperature.

Statement 25: The method of Statement 17, wherein the patient input data is obtained directly from the measuring device, from a monitoring system or over a LAN network.

Statement 26: The method of Statement 17 further comprising automatically weaning oxygen and facilitating eventual cessation of oxygen delivery.

Statement 27: The method of Statement 26, wherein adequate oxygen saturation of the patient is maintained by automatic adjustments in oxygen delivery.

Statement 28: The method of Statement 17, wherein the delivery system may be modulated internally or by manual adjustment, and wherein the delivery system may be manually overridden.

Statement 29: The method of Statement 17 further comprising receiving oxygen from a system selected from a wall output, oxygen cylinder compressor and a reservoir.

Statement 30: The method of Statement 17, wherein oxygen is delivered to the patient by a nasal cannula or a facemask.

Statement 31: The method of Statement 17, further comprising identifying and correcting input data artifacts.

Statement 32: The method of Statement 23, wherein the heart rate from the pulse oximeter is used to assess the validity of the oxygen saturation data.

Statement 33: The method of Statement 17, wherein the amount of oxygen delivered to the patient is increased or decreased by a delivery system is selected from at least one valve, at least one motor, a solenoid system, and combinations thereof.

Statement 34: The method of Statement 17 further comprising alerting the user of adverse events selected from prolonged oxygen saturation in the hypoxic range or hyperoxic range, expiratory CO₂ levels outside the target range, loss of data signal, presence of data artifacts, loss or changes to network connection, and mechanical dysfunction of the device.

Statement 35: The method of Statement 34, wherein loss of data signal may correspond to the pulse oximeter or transcutaneous membrane losing contact with the patient.

What is claimed is:

1. A device for automatically adjusting the flow of oxygen delivered to a patient setting comprising:
a microcontroller comprising:
a processing module for processing patient input data comprising oxygen saturation data;
an adjustment module for calculating the adjustment of oxygen output to the patient, wherein the adjustment of oxygen is calculated using the patient input data of the processing module;
an alarm module for sounding an alarm if the oxygen saturation data fall below a threshold;
a delivery system for automatically incrementally increasing or decreasing the amount of oxygen delivered to the patient by modifying the flow of oxygen to the patient and subsequently inspired fraction of oxygen, wherein the amount of oxygen delivered is based on the adjustment of oxygen calculated in the adjustment module;
a user interface operatively connected to the microcontroller; and
a secondary device connector operative to connect to a secondary device.

2. The device of claim 1, wherein the secondary device connector is selected from a physical connector and a wireless network connector.

3. The device of claim 1, wherein the secondary device is a monitoring system, a computer, a tablet, or a mobile device.

4. The device of claim 1, wherein the microcontroller further comprises a correction module for the identification and correction of artifacts in the patient input data.

5. The device of claim 1, wherein the oxygen saturation data is obtained from a pulse oximeter or transcutaneous membrane.

6. The device of claim 5, wherein the patient's heart rate is obtained from the pulse oximeter.

7. The device of claim 1, wherein the patient input data further comprises heart rate, respiratory rate, expiratory CO₂ levels, blood pressure, and/or temperature.

8. The device of claim 1, wherein the patient input data is obtained directly from the measuring device, from a monitoring system, or over a LAN network.

9. The device of claim 1, wherein the delivery system provides automatic oxygen weaning and facilitates eventual cessation of oxygen delivery.

10. The device of claim 9, wherein adequate oxygen saturation of the patient is maintained by automatic adjustments in oxygen delivery.

11. The device of claim 1, wherein the delivery system comprises at least one valve, at least one motor, and/or a solenoid system.

12. The device of claim 1, wherein the delivery system is modulated internally or by manual adjustment.

13. The device of claim 12, wherein the delivery system can be manually overridden.

14. The device of claim 1, wherein the device receives oxygen from a system selected from a wall output, oxygen cylinder compressor, and/or a reservoir.

15. The device of claim 1, wherein oxygen is delivered to the patient by a nasal cannula or a facemask.

16. The device of claim 1, wherein the user interface comprises:
a user input mechanism operably connected to the microcontroller;
a graphical representation of the patient's oxygen saturation and device oxygen output over time;
a graphical representation of patient physiologic data;
a graphical representation of patient data in real time; and
a graphical representation of historical data in order to monitor the course of a patient's care or the device's performance over time.

17. The device of claim 16, wherein the user input mechanism is configured for entering or altering the patient input data, the adjustment of oxygen, the threshold, and/or the amount of oxygen delivered to the patient.
